(19) United States
(12) Patent Application Publication
(10) Pub. No.: US 2010/0239514 A1
(43) Pub. Date: Sep. 23, 2010

(54) COMPOSITIONS AND METHODS FOR USE ON ARTIFICIALLY COLORED HAIR

(75) Inventors: Padmaja Prem, Saddle Brook, NJ (US); Aziza Suleiman, Paterson, NJ (US); Marie Huynh, Monmouth Junction, NJ (US); Frederic Cervantes, Westfield, NJ (US)

Correspondence Address: L'Oreal USA;Patent Department, 30 L'Oreal Way, Clark, NJ, 07066, US

(73) Assignee: L:Oreal, Paris (FR)

(21) Appl. No.: 12/679,588

(22) PCT Filed: Oct. 16, 2008
(86) PCT No.: PCT/US08/80141

§ 371 Date: Mar. 23, 2010

Related U.S. Application Data
(60) Provisional application No. 60/981,274, filed on Oct. 19, 2007.

(51) Int. Cl. A61K 8/89; A61Q 5/06; A61Q 17/04; A61Q 5/04; A61Q 5/12; A61Q 5/10

(52) U.S. Cl. 424/70.2; 424/70.12; 424/70.6; 424/70.9; 8/405

(57) ABSTRACT

The present invention provides a cosmetic composition comprising at least one saccharide-siloxane copolymer, a cosmetically-acceptable medium, and optionally, at least one auxiliary agent chosen from a nonionic agent and a cationic agent, wherein the pH of the composition is up to about 5.8. The present invention also provides for a method of using said composition for enhancing color protection of artificially colored hair while providing color vibrancy, excellent conditioning, natural feel and brilliant shine.

### STATEMENT OF RELATED APPLICATIONS

This application is a national phase of PCT/US08/80141, filed on Oct. 16, 2008 which claims priority to U.S. Provisional Application No. 60/981,274, filed Oct. 19, 2007, the entire contents of all are hereby incorporated by reference.

### FIELD OF THE INVENTION

The present invention relates to compositions and methods for enhancing color protection of artificially colored hair while providing color vibrancy, excellent conditioning, natural feel and brilliant shine.

### BACKGROUND OF THE INVENTION

Hair is composed of keratinous fibers and is inclusive of head hair, eyebrows, eyelashes, mustache, beard, and other types of body hair. Hair is commonly dyed with various coloring agents. Such hair coloring agents often fade with time due to washing or upon exposure to environmental factors such as sun, humidity, and pollution. This leads to a brassy, dull appearance and results in more frequent re—coloring than desired, which may result in less conditioned hair.

One common method of helping to prevent color fading in artificially colored hair is to employ a deep conditioner containing cationic agents and silicones, which are believed to protect artificial hair color and/or condition the hair fibers.

It has been surprisingly discovered that the use of cosmetic compositions containing a saccharide-siloxane copolymer, wherein the composition has a pH of up to about 5.8, results in enhanced color protection of artificially colored hair while also providing color vibrancy, excellent conditioning, natural feel and brilliant shine.

### SUMMARY OF THE INVENTION

The present invention is directed to a method of enhancing color protection of artificially colored hair comprising applying onto the hair a cosmetic composition containing:

a) at least one saccharide-siloxane copolymer;
b) a cosmetically acceptable medium; and,
c) optionally, at least one auxiliary agent chosen from a nonionic agent and a cationic agent,
wherein said composition has a pH of up to about 5.8.

The present invention is also directed to a composition for enhancing color protection of artificially colored hair comprising:

a) at least one saccharide-siloxane copolymer;
b) a cosmetically acceptable medium; and,
c) optionally, at least one auxiliary agent chosen from a nonionic agent and a cationic agent,
wherein said composition has a pH of up to about 5.8.

The present invention is also directed to a method of imparting color vibrancy onto artificially colored hair comprising applying onto the hair a cosmetic composition containing:
(a) at least one saccharide-siloxane copolymer;
(b) a cosmetically acceptable medium; and,
(c) optionally, at least one auxiliary agent chosen from a nonionic agent and a cationic agent,
   wherein said composition has a pH of up to about 5.8.

### DETAILED DESCRIPTION OF THE INVENTION

Other than in the operating examples, or where otherwise indicated, all numbers expressing quantities of ingredients and/or reaction conditions are to be understood as being modified in all instances by the term “about”.

As used herein, the expression “at least one” means one or more and thus includes individual components as well as mixtures/combinations.

“Cosmetically acceptable” means that the item in question is compatible with any keratin material. For example, “cosmetically acceptable medium” means a medium that is compatible with any keratin material.

“Keratin material” includes, for example, skin, hair, nails, eyelashes, eyelids, eyebrows, lips and any other area of body or facial skin.

It has now been surprisingly and unexpectedly found that compositions comprising at least one saccharide-siloxane copolymer in combination with a cosmetically acceptable medium, wherein said composition has a pH of up to about 5.8, provide enhanced color protection to artificially colored hair while also providing color vibrancy, conditioning, feel and shine to the hair.

A. Saccharide-Siloxane Copolymer

The compositions may contain at least one saccharide-siloxane copolymer. Various synthetic routes to suitable saccharide-siloxane copolymers are well known in the art and may be employed. One of ordinary skill in the art will appreciate that suitable saccharide-siloxanes may be formed from a variety of synthetic means and that the saccharide may be covalently linked to the siloxane through a variety of linking bonds described below.

The saccharide-siloxane copolymer has the following structure:

Y—X—S—X—Y

Wherein Y is a hydroxyl-functional substituted or unsubstituted saccharide bonded to the organopolysiloxane group, S, through linking group, X. According to one aspect of the present invention, the hydroxyl-functional saccharide comprises an aldonic acid or an oligoaldonic acid. In a more specific embodiment the aldonic acid or the oligoaldonic acid comprises a lactone. Two exemplary lactones include gluconolactone and lactobionolactone.

Other hydroxyl-functional saccharides which may be used include a) monosaccharide units such as glucopyranose (glucose), mannose, allose, altrose, galactose, idose, talose, gulose, ribose, arabinose, xylose, fructose, fucose, N-acetylglucosamine, N-acetylgalactosamine, sialic acid, and esters of the preceding, and b) polysaccharide units such as cellulose, amulose, and their esters. Gluconolactone is preferred among the preceding as the hydroxyl-functional saccharide.

The organopolysiloxane group, S, along with linking group X, can be specifically exemplified as:

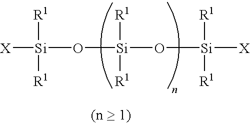

Wherein R¹ is a C₁ to C₁₀ substituted or unsubstituted linear or branched alkyl or aryl groups. The alkyl can be exemplified by methyl, ethyl, propyl, butyl, pentyl, isopropyl, isobutyl, cyclopentyl, and cyclohexyl, while the aryl can be exemplified by phenyl and naphthyl. Methyl is preferred among the preceding for R¹.

The linking group X may be chosen from an alkyl, amide, amino, urethane, urea, ester, ether, thioether, epoxide, or acetal functional linking group. A secondary amino is preferred among the preceding as the linking group.

An example of a suitable saccharide-siloxane copolymer is Gluconamidoethylaminopropyl Silicone sold by Dow Corning under the product name CE-8810 SUGAR SILICONE EMULSION.

The saccharide-siloxane copolymer may be present in the inventive composition in an amount of from about 0.1% to about 60.0% by weight, based on the total weight of the composition. The saccharide-siloxane copolymer is typically present in the inventive composition in an amount of from about 0.1% to about 47.5% by weight, preferably from about 1% to about 20% by weight, and more preferably from about 1% to about 10% by weight, based on the total weight of the composition.

B. Cosmetically Acceptable Medium

The compositions of the present invention can be formulated into or with any cosmetically acceptable carrier or diluent. Examples of such carriers or diluents are water, alcohols, polyols, and oils such as, for example, hydrocarbon oils and silicone oils. The carrier or diluent is typically present in the composition in an amount of from about 50% to about 90% by weight, preferably from about 60% to about 80% by weight, and more preferably from about 70% to about 80% by weight, based on the total weight of the composition.

C. Cationic Agent

The cosmetic composition may further comprise at least one cationic agent such that, if present in the composition, the total charge of the composition is less than about 0.10 meq/g, preferably less than about 0.05 meq/g, preferably less than about 0.02 meq/g. The at least one cationic agent may be chosen from a cationic polymer and a cationic surfactant.

i. Cationic Polymer

Among the cationic polymers that may be used, non-limiting mention may be made of those containing units comprising primary, secondary, tertiary and quaternary amine groups, which may form part of the main macromolecular chain and/or may be carried by at least one side group directly connected to the main macromolecular chain.

The cationic polymers may, for example, be chosen from polymers of the polyquaternary amine type, polymers of the polyquaternary aminoamide type, and polymers of the polyquaternary ammonium type.

For example, the synthetic cationic polymers may be chosen from polymers of the following families:

(1) homopolymers and copolymers of acrylic esters, methacrylic esters, acrylamides and methacrylamides comprising at least one amine functional group.

The copolymers of the family (1) can further comprise at least one unit deriving from comonomers which may be chosen from acrylamides, methacrylamides, diacetone acrylamides, acrylamides and methacrylamides substituted on the nitrogen atom by at least one group chosen from lower C₁₋₄ alkyl groups, groups derived from acrylic and methacrylic acids and from their esters, vinyllactams, such as vinylpyrrolidone and vinylcaprolactam, and vinyl esters.

For example, the copolymers of the family (1) may be chosen from:
copolymers of acrylamide and of dimethylaminoethyl methacrylate which may be quaternized with dimethyl sulfate or with a dimethyl halide, such as that sold under the name Hercofloc® by Hercules,
copolymers of acrylamide and of methacryloyloxyethyltrimethylammonium chloride, disclosed, for example, in Patent Application No. EP-A-080 976 and sold under the name Binaquat® P 100 by Ciba-Geigy,
copolymers of acrylamide and of methacryloyloxyethyltrimethylammonium methyl sulfate sold under the name Reten® by Hercules,
vinylpyrrolidone/dialkylaminoalkyl acrylate and methacrylate copolymers, which may or may not be quaternized, such as the products sold under the name Gafquat® by ISP, for example, Gafquat® 734 and Gafquat® 755, and the products named Copolymer 845, 958 and 937. These polymers are disclosed in detail in French Patent Nos. 2 077 143 and 2 393 573,
dimethylaminoethyl methacrylate/vinylcaprolactam/vinylpyrrolidone terpolymers, such as the product sold under the name Gaffix® VC 713 by ISP,
vinylpyrrolidone/methacrylamidopropyldimethylamine copolymers, sold, for example, under the name Styleze® CC 10 by ISP, and
vinylpyrrolidone/quaternized dimethylaminopropylmethacrylamide copolymers, such as the product sold under the name Gafquat® HS 100 by ISP;

(2) Polymers comprising at least one piperazinyl unit and at least one unit chosen from alkylene and hydroxyalkylene units, wherein the alkylene and hydroxyalkylene units comprise at least one group chosen from straight- and branched-chain alkylene and hydroxyalkylene groups respectively, optionally interrupted by at least one entity chosen from oxygen, sulfur and nitrogen atoms, aromatic rings, and heterocyclic rings; and the oxidation and/or quaternization products of these polymers.

(3) Water-soluble polyaminoamides prepared, for example, by polycondensation of an acidic compound with a polyamine. These polyaminoamides can be crosslinked by at least one crosslinking agent chosen from epihalohydrins, diepoxides, dianhydrides, unsaturated dianhydrides, bisunsaturated derivatives, bishalohydrins, bisazetidiniums, bishaloacyldiamines, alkyl bishalides, and oligomers resulting from the reaction of a bifunctional compound reactive with respect to a bishalohydrin, a bisazetidinium, a bishaloacyldiamine, an alkyl bishalide, an epihalohydrin, a diepoxide and a bisunsaturated derivative; wherein the at least one crosslinking agent is used in an amount ranging from 0.025 to 0.35 mol per amine group of the polyaminoamide. These polyaminoamides can be alkylated or, if they comprise at least one tertiary amine functional group, they can be quaternized. Such polymers are disclosed, for example, in French Patent Nos. 2 252 840 and 2 368 508;

(4) Polyaminoamide derivatives resulting from the condensation of polyalkylenepolyamines with polycarboxylic acids, followed by an alkylation by bifunctional agents. The polyaminoamide derivatives may, for example, be chosen from adipic acid/dialkylaminohydroxyalkyl/dialkylenetriamine polymers wherein the alkyl group comprises from 1 to 4 carbon atoms and, for example, is chosen from methyl, ethyl and propyl groups and alkylene groups comprising from 1 to 4 carbon atoms and, for example, is chosen from ethylene groups.

(5) Polymers obtained by reaction of a polyalkylenepolyamine comprising two primary amine groups and at least one secondary amine group with a dicarboxylic acid chosen from diglycolic acid and saturated aliphatic dicarboxylic acids comprising from 3 to 8 carbon atoms, wherein the molar ratio of the polyalkylenepolyamine to the dicarboxylic acid ranges from 0.8:1 to 1.4:1. The polyaminoamide resulting from this reaction is subsequently brought to react with epichlorohydrin in a molar ratio of epichlorohydrin in relation to the secondary amine group of the polyaminoamide ranging from 0.5:1 to 1.8:1.

For example, polymers of this type are sold under the name Hercosett® 57 by Hercules Inc. and under the name of PD 170 and Delsette® 101 by Hercules in the case of the adipic acid/epoxypropyl/diethylenetriamine copolymer;

(6) Cyclopolymers of alkyldiallylamine and of dialkyldiallylammonium. For example, the cyclopolymers of alkyldiallylamine and of diallylammonium may be chosen from the homopolymers of dimethyldiallylammonium chloride sold under the name Merquat® 100 by Nalco (and its homologs of low weight-average molecular masses) and the copolymers of diallyldimethylammonium chloride and of acrylamide sold under the name Merquat® 550;

(7) Polymeric quaternary ammonium silicone compounds and their salts, such as Silicone Quaternium-16 sold as DC5-7113 by the company Dow Corning.

(8) Diquaternary ammonium polymers comprising repeating units. These polymers are disclosed, for example, in French Patent No. 4,027,020;

(9) Polyquaternary ammonium polymers comprising repeating units. For example, the polyquaternary ammonium polymers comprising repeating units may be chosen from the products Mirapol® A 15, Mirapol® AD1, Mirapol® AZ1 and Mirapol® 175, sold by Miranol;

(10) Quaternary polymers of vinylpyrrolidone and of vinylimidazole, for example, the products sold under the names Luviquat® FC 905, FC 550 and FC 370 by BASF. For example, the quaternary polymers of vinylpyrrolidone and of vinylimidazole may be chosen from copolymers of vinylpyrrolidone and of methylvinylimidazolium chloride;

(11) Amines and Polyamines, such as Lupamin 9095 (polyvinyl amine) by the company BASF. Another example is Polyquart® H sold by Henkel, which is referenced under the name of Polyethylene Glycol (15) Tallow Polyamine in the CTFA dictionary. Amidoamine derivatives may also be chosen, such as Stearamidopropyl Dimethylamine;

(12) Crosslinked and noncrosslinked polymers of methacryloyloxy(C₁₋₄)alkyltri(C₁₋₄)alkylammonium salts, such as the polymers obtained by homopolymerization of dimethylaminoethyl methacrylate quaternized by methyl chloride or by copolymerization of acrylamide and of dimethylaminoethyl methacrylate quaternized by methyl chloride, the homopolymerization or the copolymerization being followed by a crosslinking by a compound possessing olefinic unsaturation, for example, methylenebisacrylamide. It is also possible to use, for example, a crosslinked acrylamide/methacryloyloxyethyltrimethylammonium chloride (20/80 by weight) copolymer in the form of a dispersion comprising 50% by weight of said copolymer in mineral oil. This dispersion is sold under the name of Salcare® SC 92 by Ciba. It is also possible to use a crosslinked homopolymer of methacryloyloxyethyltrimethylammonium chloride comprising approximately 50% by weight of the homopolymer in mineral oil or in a liquid ester. These dispersions are sold under the names of Salcare® SC 95 and Salcare® SC 96 by Ciba; and,

(13) Cationic polysaccharide polymers, such as, (a) The cellulose ether derivatives comprising at least one quaternary ammonium group, such as polymers sold under the “JR” (JR 400, JR 125, JR 30M) and “LR” (LR 400, LR 30M) names by Almerchol. These polymers are also defined in the CTFA dictionary as quaternary ammoniums of hydroxyethylcellulose having reacted with an epoxide substituted by a trimethylammonium group;

(b) Cationic derivatives of celluose, such as copolymers of cellulose and derivatives of cellulose which are grafted with at least one water-soluble quaternary ammonium monomer. Examples are hydroxyalkylcelluloses, for example hydroxymethyl-, hydroxyethyl- and hydroxypropylcelluloses, grafted, for example, with at least one salt chosen from methacryloylethyltrimethylammonium, methacrylamidopropyltrimethylammonium and dimethyldiallylammonium salts.

The commercially available products corresponding to this definition are, for example, the products sold under the name Celquat® L 200 and Celquat® H 100 by National Starch;

(c) Cationic polysaccharides comprising at least one trialkylammonium cationic group. The cationic polysaccharides may, for example, be chosen from guar gums modified by a 2,3-epoxypropyltrimethylammonium salt, for example, the chloride. Such products are sold, for example, under the tradenames of Jaguar® C13 S, Jaguar® C 15, Jaguar® C 17 and Jaguar® C162 by Meyhall; and,
(d) Chitosans and the salts thereof, such as chitosan acetate, lactate, glutamate, gluconate and pyrrolidonecarboxylate.
For example, the chitosans and the salts thereof may be chosen from chitosans having a degree of deacetylation of 90.5% by weight sold under the name Kytan Brut Standard by Aber Technologies and the chitosan pyrrolidonecarboxylate sold under the name Kytamer® PC by Amerchol.

ii. Cationic Surfactant

The cationic surfactant may, for example, be chosen from:

A). Quaternary Ammonium salts. The quaternary ammonium salts that may be used include (a) tetraalkylammonium chlorides, for example dialkyldimethylammonium and alkyltrimethylammonium chlorides in which the alkyl radical has from about 12 to 22 carbon atoms, e.g., Genamin (Behentrimonium chloride) by the company Clariant, ARQUAD 16-25 LO (Cetrimonium chloride) by the company Akzo-Nobel, distearyidimethyl-ammonium, and benzyldimethylstearylammonium chloride, and (b) palmitylamidopropyltrimethylammonium chloride and stearamidopropyldimethyl(myristyl acetate)ammonium chloride sold under the name Ceraphyl® 70 by the company Van Dyk.

B) Quaternary Ammonium salts of imidazolinium. Such a product is, for example, Quaternium-27 (CTFA 1997) or Quaternium-83 (CTFA 1997), which are sold under the names “REWOQUAT” W75, W90, W75PG and W75EPG by the company Witco;

C) Diquaternary Ammonium salts. Such diquaternary ammonium salts, for example, include propanetallowediammonium dichloride; and,

D) Quaternary Ammonium salts comprising at least one ester functional group. Such compounds are sold, for example, under the names DEHYQUART by the company Cognis, STEPANQUAT by the company Stepan, NOXAMIUM by the company Ceca, and REWOQUAT WE 18 by the company Rewo-Witco.

Preferred cationic agents for this invention may be chosen from quaternary ammonium compounds with single chain or double fatty chains such as Genamin (Behentrimonium chloride) by the company Clariant or ARQUAD 16-25 LO (Cetrimonium chloride) by the company Akzo-Nobel, quaternary esters such as DEHYQUART F75 by the company Cognis, silicone quaternium compounds such as DC5-7113 (Silicone Quaternium-16) by the company Dow Corning, and amines or polyamines, such as MACKINE 301 (Stearamidopropyl Dimethylamine) by the company Mac-Intyre or Lupamin 9095 (polyvinyl amine) by the company BASF.

D. Non-Ionic Agent(s)

The cosmetic composition may further comprise at least one non-ionic agent. The at least one non-ionic agent may be chosen from a non-ionic thickening polymer and a non-ionic surfactant.
Suitable nonionic thickening polymers, also known as “rheology modifiers”, may be chosen from fatty acid amides, for example, coconut monoethanolamide, coconut diethanolamide, and oxyethylenated carboxylic acid alkyl ether monoethanolamide; cellulose-based thickeners, such as hydroxymethylcellulose, hydroxyethylcellulose, hydroxypropylcellulose, and hydroxypropylmethylcellulose; guar gum and its derivatives, for instance, the hydroxypropyl guar gum sold under the name JAGUAR HP105 by the company Rhodia; gums of microbial origin, for example, xanthan gum and scleroglucan gum); and nonionic associative polymers.

Non-limiting examples of nonionic associative polymers may include:

(1) celluloses modified with groups comprising at least one fatty chain, for example:
hydroxyethylcelluloses modified with groups comprising at least one fatty chain, such as alkyl, arylalkyl, and alkylaryl groups, and mixtures thereof, wherein the alkyl groups may be C₈-C₂₂ alkyl groups, for instance, the product Natrosol Plus Grade 330 CSO(C₁₋₆ alkyls) sold by the company Aqualon, the product Bermocoll EHM 1008 sold by the company Berol Nobel, the product Methocel (hydroxylpropyl cellulose) sold from Dow/Amerchol, the product Klucel (hydroxylpropryl methyl cellulose) sold from Hercules, and,
celluloses modified with alkylphenyl polyalkylene glycol ether groups, such as the product Amercell Polymer HM-1500® (nonylphenyl polyethylene glycol (15) ether) sold by the company Amerchol;

(2) hydroxypropyl guars modified with groups comprising at least one fatty chain, such as the product Esaflor HM 22® (C₂₂ alkyl chain) sold by the company Lamberti, and the products RE210-18® (C₁₄ alkyl chain) and RE205-1® (C₂₀ alkyl chain) sold by the company Rhone-Poulenc;

(3) copolymers of vinylpyrrolidone and of fatty-chain hydrophobic monomers; for example:
the products Antaron V216® or Ganex V216® (vinylpyrrolidone/hexadecene copolymer) sold by the company T.S.P; and
the products Antaron V220® or Ganex V220® (vinylpyrrolidone/eicosene copolymer) sold by the company I.S.P;

(4) copolymers of C₁-C₆ alkyl methacrylates or acrylates and of amphiphilic monomers comprising at least one fatty chain, for example, the oxyethylenated methyl acrylate/stearyl acrylate copolymer sold by the company Goldschmidt under the name Antil 208®;

(5) copolymers of hydrophilic methacrylates or acrylates and of hydrophobic monomers comprising at least one fatty chain, for example, polyethylene glycol methacrylate/lauryl methacrylate copolymer.

(6) polyurethane polyethers comprising in their chain both hydrophilic blocks, for example, polyoxyethylenated blocks, and hydrophobic blocks, which may be aliphatic sequences alone and/or cycloaliphatic and/or aromatic sequences. Examples of such polyurethane polyethers include those sold by Rohm & Haas under the names Aculyn 44® and Aculyn 46®. Aculyn 46® is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%). Aculyn 44® is a polycondensate of polyethylene glycol containing 150 or 180 mol of ethylene oxide, of decyl alcohol and of methylenebis(4-cyclohexyl-isocyanate) (SMDI), at 35% by weight in a mixture of propylene glycol (39%) and water (26%);

(7) polymers with an aminoplast ether skeleton containing at least one fatty chain, such as the Pure Thix® compounds sold by the company Sud-Chemie.

Non-limiting examples of non-ionic surfactants may include alkoxylated derivatives of the following: fatty alcohols, alkyl phenols, fatty acids, fatty acid esters and fatty acid amides, wherein the alkyl chain is in the C₁₂₋₅₀ range, typically in the C₁₆₋₄₀ range, more typically in the C₂₄ to C₄₀ range, and having from about 1 to about 110 alkoxy groups. The alkoxy groups are selected from the group consisting of C₂-C₆ oxides and their mixtures, with ethylene oxide, propylene oxide, and their mixtures being the typical alkoxides. The alkyl chain may be linear, branched, saturated, or unsaturated. Of these alkoxylated non-ionic surfactants, the alkoxylated alcohols are typical, and the ethoxylated alcohols and propoxylated alcohols are more typical. The alkoxylated alcohols may be used alone or in mixtures with those alkoxylated materials disclosed herein-above.

Other representative examples of such ethoxylated fatty alcohols include laureth-3 (a lauryl ethoxylate having an average degree of ethoxylation of 3), laureth-23 (a lauryl ethoxylate having an average degree of ethoxylation of 23), ceteth-10 (a cetyl alcohol ethoxylate having an average degree of ethoxylation of 10), steareth-10 (a stearyl alcohol ethoxylate having an average degree of ethoxylation of 10), steareth-2 (a stearyl alcohol ethoxylate having an average degree of ethoxylation of 2), steareth-100 (a stearyl alcohol ethoxylate having an average degree of ethoxylation of 100), beheneth-5 (a behenyl alcohol ethoxylate having an average degree of ethoxylation of 5), beheneth-10 (a behenyl alcohol ethoxylate having an average degree of ethoxylation of 10), and other derivatives and mixtures of the preceding.

Commercially available nonionic surfactants are Brij® nonionic surfactants from Uniqema, Willmington, Del. Typically, Brij® is the condensation products of aliphatic alcohols with from about 1 to about 54 moles of ethylene oxide, the alkyl chain of the alcohol being typically a linear chain and having from about 8 to about 22 carbon atoms, for example, Brij 72 (i.e., Steareth-2) and Brij 76 (i.e., Steareth-10).

Also useful herein as nonionic surfactants are alkyl glycosides, which are the condensation products of long chain alcohols, which are the condensation products of long chain alcohols, e.g. C₈-C₃₀ alcohols, with sugar or starch polymers. These compounds can be represented by the formula (S)n-O—R wherein S is a sugar moiety such as glucose, fructose, mannose, galactose, and the like; n is an integer of from about 1 to about 1000, and R is a C₈-C₃₀ alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants are alkyl polyglucosides wherein S is a glucose moiety, R is a C₈-C₂₀ alkyl group, and n is an integer of from about 1 to about 9. Commercially available examples of these surfactants include decyl polyglucoside (available as APC® 325 CS) and lauryl polyglucoside (available as APG® 600CS and 625 CS), all the above-identified polyglucosides APG® are available from Cognis, Ambler, Pa. Also useful herein sucrose ester surfactants such as sucrose cocoate and sucrose laurate.

Other non-ionic surfactants suitable for use in the present invention are glyceryl esters and polyglyceryl esters, including but not limited to, glyceryl monesters, typically glycerly monesters of C₁₆-C₂₂ saturated, unsaturated and branched chain fatty acids such as glyceryl oleate, glyceryl monostearate, glyceryl monoisostearate, glyceryl monopalmitate, glyceryl monobehenate, and mixtures thereof, and polyglyceryl esters of C₁₆-C₂₂ saturated, unsaturated and branched chain fatty acids, such as polyglyceryl-4 isostearate, polyglyceryl-3 oleate, polyglyceryl-2 sesquioleate, triglyceryl diisostearate, diglyceryl monooleate, tetraglyceryl monooleate, and mixtures thereof.

Also useful herein as nonionic surfactants are sorbitan esters. Preferable are sorbitan esters of C₁₆-C₂₂ saturated, unsaturated and branched chain fatty acids. Because of the manner in which they are typically manufactured, these sorbitan esters usually comprise mixtures of mono-, di-, tri-, etc. esters. Representative examples of suitable sorbitan esters include sorbitan monooleate (e.g., SPAN® 80), sorbitan sesquioleate (e.g., Arlacel® 83 from Uniqema, Wilmington, Del.), sorbitan monoisostearate (e.g., GRILL® 6 from Croda, Inc., Edison, N.J.), sorbitan stearates (e.g., SPAN® 60), sorbitan trioleate (e.g., SPAN® 85), sorbitan tristearate (e.g., SPAN® 65), sorbitan dipalmitates (e.g., SPAN® 40), and sorbitan isostearate. Sorbitan monoisostearate and sorbitan sesquioleate are particularly preferred emulsifiers for use in the present invention.

Also suitable for use as nonionic surfactants are alkoxylated derivatives of glyceryl esters, sorbitan esters, and alkyl polyglycosides, wherein the alkoxy groups is selected from the group consisting of C₂-C₆ oxides and their mixtures, with ethoxylated or propoxylated derivatives of these materials being ypical. Nonlimiting examples of commercially available ethoxylated materials include TWEEN® (ethoxylated sorbitan mono-, di- and/or tri-esters of C₁₂ to C₁₈ fatty acids with an average degree of ethoxylation of from about 2 to 20).

Suitable non-ionic agents for this invention may be chosen from glyceryl esters and polyethylene glycol esters of stearic acid, such as glyceryl stearate and PEG-100 stearate. The non-ionic agent(s) may be present in the composition in an amount of from about 0.1% to about 20.0% by weight, preferably from about 1.0% to about 10.0% by weight, and more preferably from about 2.0% to about 8.0% by weight, based on the total weight of the composition.

E. pH

The compositions of the present invention have a pH of up to about 5.8, preferably up to about 5.5, preferably up to about 5.0, preferably up to about 4.5, preferably up to about 4.0.

F. Optional Ingredients

The compositions of the present invention may further comprise one or more components known for use in hair care compositions. Examples of such components include surfactants that are suitable for use on the hair or the skin. Suitable surfactants include non-ionic surfactants, anionic surfactants, amphoteric surfactants, zwitterionic surfactants, or mixtures thereof. Surfactants useful in the invention include those described in Kirk-Othmer, Encyclopedia of Chemical Technology (4.sup.th Ed.), vol. 23, John Wiley and Sons, Inc., NY and in the C.T.F.A. International Cosmetic Ingredient Dictionary and Handbook, 11^{th} edition, vol. 3, (2006). The surfactant can be selected for its cleansing property, foaming property, lathering property, emulsifying property or other desirable property.

Other examples of optional ingredients include, but are not limited to, ultraviolet light filters, dyes, hair colorants, hair fixatives, hair waving agents, hair straightening agents, organic solvents or diluents, foam boosters, pH adjusting agents, conditioning agents, humectants, lipids, fragrances, preservatives, proteins, protein derivatives, amino acids, amino acid derivatives, skin active agents, suspending agents, sunscreens, thickeners, vitamins, ceramide, uv absorbers (e.g., benzophenone), botanicals, anti-oxidants, retinoid, anti-dandruff, anti hair-loss and viscosity adjusting agents. These and other cosmetic additives commonly used in hair care formulations are described in, for example, C.T.F.A. International Cosmetic Ingredient Dictionary and Handbook, 11^{th} edition, vol. 3, (2006).

These optional components may be present in the composition in an amount of from about 0.01% to about 10.0% by weight, preferably from about 0.01% to about 5.0% by weight, and more preferably from about 0.01% to about 2.0% by weight, based on the total weight of the composition.

The compositions of the present invention can be prepared by using various formulation and mixing methods commonly employed in the art to prepare hair care compositions, such as pre-color treatments, shampoos, conditioners, leave-in-treatments, post-color treatments, and/or hair coloring agents.

The compositions according to the present invention can be formulated into, for example, shampoos, conditioners, hair treatment creams, gels, mousse, pump hair sprays, aerosol hair sprays, set lotions, blow styling lotions, hair color lotions, hair relaxing compositions, permanent wave first agents, permanent wave second agents, and coloring compositions.

The compositions according to the present invention can be used in aqueous and anhydrous systems.

The compositions of the present invention are used by applying to the hair before, during or after the hair has been colored or dyed. The hair may be wet, dry or semi-dry. The compositions described herein can be applied to the hair by working, rubbing, spraying, or massaging the composition into the hair so that substantially all or some of the hair is contacted with the composition. In one embodiment where treatment for only a portion of the hair is needed, the composition can be applied to the localized region as needed. The composition may also be delivered onto the hair by use of an applicator or device.

According to one embodiment of the present invention, a method of enhancing color protection and color vibrancy in artificially colored hair comprising applying the hair care composition set forth above to the hair in an amount effective to enhance color protection and color vibrancy in the hair is provided.

### EXAMPLE

### Example 1

The following compositions were prepared. Compositions A-C are the inventive compositions. Composition D and E are controls, representing compositions with a total charge that is outside of the inventive range. Compositions A-E have a pH of between 4.4-5.5.

| | | | | D | E |
|---|---|---|---|---|---|
| | A | B | C | (control) | (control) |
| | wt. % | wt. % | wt. % | wt. % | wt. % |
| GLUCONAMIDOETHYL | 5.0 | 5.0 | 5.0 | 5.0 | |
| AMINOPROPYLSILICONE | | | | | |
| POLY(N-VINYLFORMAMIDE/ | 1.313 | | | | |
| VINYLAMINE) | | | | | |
| UNDECETH-11 | | 0.375 | | | |
| UNDECETH-5 | | 0.150 | | | |
| SILICONE QUATERNIUM-16 | | 1.100 | | | |
| ISOPROPYL ALCOHOL | | | 0.1134 | 1.125 | 1.125 |
| BEHENTRIMONIUM | | | 0.4977 | 4.9375 | 4.9375 |
| CHLORIDE | | | | | |
| CENTRIMONIUM | | | | | 0.03 |
| CHLORIDE | | | | | |
| TRIDECETH-12 | | | | | 0.15 |
| GLYCERIN | | | | | 5.00 |
| CETEARYL ALCOHOL | | | | | 6.00 |
| AMODIMETHICONE | | | | | 1.75 |
| CETYL ESTERS | | | | | 1.50 |
| EUPHORBIA CERIFERA | | | | | 2.00 |
| (CANDELILLA) WAX | | | | | |
| MYRISTYL ALCOHOL | 0.05 | 0.05 | 0.05 | 0.05 | |
| STEARYL ALCOHOL | 2.55 | 2.55 | 2.55 | 2.55 | |
| PHENOXYETHANOL | 0.10526 | 0.10526 | 0.10526 | 0.10526 | |
| GLYCERYL STEARATE | 3.0 | 3.0 | 2.0 | 2.0 | |
| CETYL ALCOHOL | 1.9 | 1.9 | 1.9 | 1.9 | |
| CHLORHEXIDINE | 0.05 | 0.05 | 0.05 | 0.05 | 0.03 |
| DIHYDROCHLORIDE | | | | | |
| BUTYLOCTANOL | 0.5263 | 0.5263 | 0.5263 | 0.5263 | |
| IODOPROPYNYL | 0.005263 | 0.005263 | 0.005263 | 0.005263 | |
| BUTYLCARBAMATE | | | | | |
| ALCOHOL | 0.10526 | 0.10526 | 0.10526 | 0.10526 | |
| C11-15 PARETH-40 | 0.5263 | 0.5263 | 0.5263 | 0.5263 | |
| HYDROXYPROPYLCELLULOSE | | | 1.5 | 1.5 | |
| PEG-100 STEARATE | 3.0 | 3.0 | 2 | 2 | |
| METHYLPARABEN | 0.3 | 0.3 | 0.3 | 0.3 | 0.3 |
| FRAGRANCE | | | | | 0.7 |
| Q.S. | 100.00 | 100.00 | 100.00 | 100.00 | 100.00 |

The ability of each composition to enhance color protection was then determined using the following protocol.

Swatches of 90% Grey Hair were artificially colored using a composition containing a mixture of dyes. Immediately after coloring, the hair was rinsed with water and towel dried. Conditioning treatment compositions A-E were applied to swatches. After approximately 3 minutes, the swatches were rinsed, blow-dried, and initial color measurements on the hair was taken for each swatch. Next, the conditioner treated hair swatches were shampooed and blow-dried. Treatment compositions A-E were applied to the swatches for approximately 3 minutes. The swatches were then rinsed with water and blow-dried. This shampooing, rinsing, drying, conditioning, rinsing, and drying process consisted on one cycle. This shampoo-wash cycle was repeated for up to a total of twelve cycles. Colorimetric measurements of the treated hair swatches were taken at the fifth, eighth and twelfth shampoo-wash cycles.

The color on each of the hair swatches was determined using the CIELAB L*a*b* system using a Sphere Spectrophotometer SP60 Series. Six measurements were taken on each swatch, three on each side of the swatch, at the top, middle and bottom of each side. Specifically, ΔE, a measurement of color change, was calculated for each of the compositions (between treatment and standard) according to the following formula:

ΔE=\sqrt{(L*₁−L*₀)²+(a*₁−a*₀)+(b*₁−b*₀)²}

where L*₀, a*₀, and b*₀ are coordinates associated with a standard (intial color measurements on artificially colored hair which was not shampooed) and L*₁, a*₁, and b*₁ are coordinates for the artificially colored hair treated with Compositions A-E and five or eight or twelve shampoo-wash cycles.

The following results were obtained.

| | | ΔE | ΔE | ΔE |
|---|---|---|---|---|
| | | After 5 | After 8 | After 12 |
| | Total | shampoo- | shampoo- | shampoo- |
| | Charge | wash | wash | wash |
| Composition | (meq/g)* | cycles | cycles | cycles |
| A | 0.011 | 2.47 | 4.32 | 5.95 |
| B | 0.019 | 2.13 | 4.06 | 5.46 |
| C | 0.006 | 2.0 | 4.09 | 5.25 |
| D (control) | 0.129 | 3.32 | 5.68 | 7.32 |
| E (control) | 0.134 | 3.6 | 5.10 | 7.38 |

The cationic charge was obtained using a potentiometer, whereby the active cationic substance is dissolved in a Methylisobutylketone (MIRK)/Ethanol

mixture (1:1) under heat and titrated by potentiometry in acid medium using Sodium

Lauryl Sulfate (titrated anionic solution) and an electrode.

The data above shows there is a general correlation between the total available charge and the amount of color protection (ΔE) in artificially colored hair. Formulas A, B, and C, which have lower charges, show a significant improvement in color protection as evidenced by the lower ΔE values as compared to the controls. A lower ΔE value is associated with less color change between the initial color measurement and the color measurement taken after treating the swatch. The differences in color vibrancy were also visually perceivable on the swatches. Swatches treated with the inventive compositions having lower charges showed enhanced color vibrancy based on visual observations as compared to the control.

1. A method of enhancing color protection in artificially colored hair comprising applying onto the hair a cosmetic composition containing:
(a) at least one saccharide-siloxane copolymer;
(b) a cosmetically acceptable medium; and,
(c) optionally, at least one auxiliary agent chosen from a nonionic agent and a cationic agent,
wherein said composition has a pH of up to about 5.8.

2. The method of claim 1, wherein the at least one saccharide-siloxane copolymer is present in an amount from about 0.1% to about 60.0% by weight, based on the total weight of the composition.

3. The method of claim 1, wherein the at least one saccharide-siloxane copolymer is present in an amount from about 0.1% to about 47.5% by weight, based on the total weight of the composition.

4. The method of claim 1, wherein the at least one saccharide-siloxane copolymer is present in an amount from about 1.0% to about 10.0% by weight, based on the total weight of the composition.

5. The method of claim 1, wherein (c) is a cationic agent.

6. The method of claim 5, wherein the total charge of the composition is less than about 0.10 meq/g.

7. The method of claim 5, wherein the at least one cationic agent is present in an amount from about 0.001% to about 10% by weight, based on the total weight of the composition.

8. The method of claim 1, wherein (c) is a nonionic agent.

9. The method of claim 8, wherein the nonionic agent is present in an amount from about 0.1% to about 20.0% by weight, based on the total weight of the composition.

10. The method of claim 1, wherein the cosmetically acceptable medium is chosen from water, alcohols, polyols, and oils.

11. The method of claim 1, wherein said cosmetic composition further comprises at least one optional ingredient chosen from ultraviolet light filters, dyes, hair colorants, hair fixatives, hair waving agents, hair straightening agents, organic solvents or diluents, foam boosters, pH adjusting agents, conditioning agents, humectants, lipids, fragrances, preservatives, proteins, protein derivatives, amino acids, amino acid derivatives, skin active agents, suspending agents, sunscreens, thickeners, vitamins, ceramide, uv absorbers, botanicals, anti-oxidants, retinoids, anti-dandruff agents, anti hair-loss agents and viscosity adjusting agents.

12. A composition for enhancing the color protection in artificially colored hair, comprising:
(a) at least one saccharide-siloxane copolymer;
(b) a cosmetically acceptable medium; and,
(c) optionally, at least one auxiliary agent chosen from a nonionic agent and a cationic agent,
wherein said composition has a pH of up to about 5.8.

13. The composition of claim 12, wherein the at least one saccharide-siloxane copolymer is present in an amount from about 0.1% to about 60.0% by weight, based on the total weight of the composition.

14. The composition of claim 12, wherein the at least one saccharide-siloxane copolymer is present in an amount from about 0.1% to about 47.5% by weight, based on the total weight of the composition.

15. The composition of claim 12, wherein the at least one saccharide-siloxane copolymer is present in an amount from about 1.0% to about 10.0% by weight, based on the total weight of the composition.

16. The composition of claim 12, wherein (c) is a cationic agent.

17. The composition of claim 16, wherein the total charge of the composition is less than about 0.10 meq/g.

18. The composition of claim 16, wherein the at least one cationic agent is present in an amount from about 0.001% to about 10% by weight, based on the total weight of the composition.

19. The composition of claim 12, wherein (c) is a nonionic agent.

20. The composition of claim 19, wherein the nonionic agent is present in an amount from about 0.1% to about 20.0% by weight, based on the total weight of the composition.

21. The composition of claim 12, wherein the cosmetically acceptable medium is chosen from water, alcohols, polyols, and oils.

22. The composition of claim 12, wherein said cosmetic composition further comprises at least one optional ingredient chosen from ultraviolet light filters, dyes, hair colorants, hair fixatives, hair waving agents, hair straightening agents, organic solvents or diluents, foam boosters, pH adjusting agents, conditioning agents, humectants, lipids, fragrances, preservatives, proteins, protein derivatives, amino acids, amino acid derivatives, skin active agents, suspending agents, sunscreens, thickeners, vitamins, ceramide, uv absorbers, botanicals, anti-oxidants, retinoids, anti-dandruff agents, anti hair-loss agents and viscosity adjusting agents.

23. The composition of claim 12, wherein said cosmetic composition is a hair cosmetic chosen from hair cleansing products, shampoos, conditioners, leave-in-treatments, post-color treatments, hair coloring agents, hair treatment creams, gels, mousse, pump hair sprays, aerosol hair sprays, set lotions, blow styling lotions, hair color lotions, hair relaxing compositions, and hair waving compositions.

24. A method of imparting color vibrancy onto artificially colored hair comprising applying onto the hair a cosmetic composition containing:
(a) at least one saccharide-siloxane copolymer;
(b) a cosmetically acceptable medium; and,
(c) optionally, at least one auxiliary agent chosen from a nonionic agent and a cationic agent,
wherein said composition has a pH of up to about 5.8.
